(19) United States
(12) Patent Application Publication
(10) Pub. No.: US 2004/0077728 A1
(43) Pub. Date: Apr. 22, 2004

(54) Retiferol derivatives and their use in the treatment of skin diseases or conditions associated with photodamage

(76) Inventors: Pierre Barbier, Rixheim (FR); Franz W Bauer, Schaijk (NL); Peter Mohr, Basel (CH); Marc Muller, St Louis (FR); Wolfgang Pirson, Weil am Rhein (DE)

Correspondence Address: Geroge W Johnston, Hoffman-La Roche Inc, 340 Kingsland Street, Nutley, NJ, 07110, US

(21) Appl. No.: 10/432,792

(22) PCT Filed: Nov. 13, 2001
(86) PCT No.: PCT/EP01/13110

(30) Foreign Application Priority Data
Nov. 22, 2000 (EP) 00125554.6

(51) Int. Cl. A61K 31/16; A61K 31/075; C07C 235/36

(52) U.S. Cl. 514/613; 514/715; 564/123; 568/670

(57) ABSTRACT

Disclosed are retiferol derivatives of formula (I): wherein X is >C═CH₂ or —CH₂—; Y and Z are independently of each other hydrogen, fluorine or hydroxy; A is —O(CH₂)₃—, —(CH₂)₂-(1,2-C₆H₄)—, —CH═CH—(1,2-C₆H₄)—, —C═C—(1,2C₆H₄)—, —(CH₂)₂—CO—, —CH₂—O—CO—, —CH₂NHCO—, or —CH₂NHCOCH₂—; R¹ is C₁-C₅-alkyl; R² and R³ are independently of each other alkyl or perfluoroalkyl; and R⁴ is hydrogen, hydroxy, C¹-C₅-alkyl or C₁-C₅-alkyl or C₁-C₅-alkoxy; the use of such compounds to treat or present hyperproliferative skin diseases such as psoriasis, basal cell carcinomas, disorders of keratinization and keratosis; neoplastic diseases; disorders of the sebaceous glands such as acne and seborrhoic dermatitis; the use of these compounds in reversing the conditions associated with photodamage, particularly for the oral or topical treatment of the skin damaged through sun exposure, the effects of wrinkling, elastosis and premature ageing, furthermore a process for the manufacture of such compounds as well as pharmaceutical compositions containing such compounds.

The invention relates to the novel retiferol derivatives of formula I:

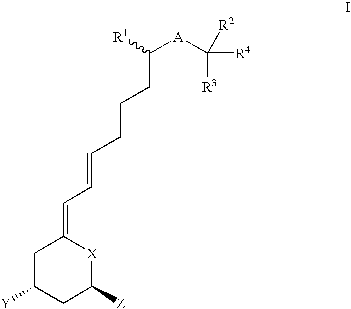

wherein

X is >C═CH₂ or —CH₂—;

Y and Z are independently of each other hydrogen, fluorine or hydroxy;

A is —O(CH₂)₃—, —(CH₂)₂-(1,2-C₆H₄)—, —CH═CH—(1,2-C₆H₄)—, —C≡C-(1,2-C₆H₄)—, —(CH₂)₂—CO—, —CH₂—O—CO—, —CH₂NHCO—, or —CH₂NHCOCH₂—;

R¹ is C₁-C₅-alkyl;

R² and R³ are independently of each other alkyl or perfluoroalkyl; and

R⁴ is hydrogen, hydroxy, C₁-C₅-alkyl or C₁-C₅-alkoxy.

The compounds of formula I can be utilized to treat or prevent hyperproliferative skin diseases such as psoriasis, basal cell carcinomas, disorders of keratinization and keratosis; neoplastic diseases; disorders of the sebaceous glands such as acne and seborrhoic dermatitis. The compounds of formula I can also be utilized in reversing the conditions associated with photodamage, particularly for the oral or topical treatment of the skin damaged through sun exposure, the effects of wrinkling, elastosis and premature ageing.

The present invention furthermore relates to a process for the preparation of compounds of formula I, to pharmaceutical compositions containing such compounds, to the use of these compounds for the treatment and prevention of the above mentioned disorders and for the manufacture of pharmaceutical compositions for the treatment and prevention of the above mentioned disorders, as well as to a method for treating and preventing such disorders.

The term “alkyl” as used herein denotes straight or branched chain alkyl residues containing 1 to 12 carbon atoms, preferably 1 to 4 carbon atoms, such as methyl, ethyl, butyl, isopropyl, isobutyl, tert.-butyl.

The term “perfluorinated alkyl” denotes alkyl groups as defined above wherein all hydrogen atoms are substituted by fluorine, such as in trifluoromethyl, pentafluoroethyl, perfluoropropyl and the like.

The term “alkoxy” as used herein are groups wherein alkyl is as defined above.

The “hydroxy protecting group” can be any conventional hydroxy protecting group. Examples of such groups are silyl ether groups such as tert.-butyl-dimethylsilyl or tert.-butyl-diphenylsilyl, such silyl protecting groups are used for the hydroxy groups in position 1 and 3 of compounds of formula I. Other examples of a hydroxy protecting group are tetrahydropyranyl (THP), methoxymethyl (MOM), or methoxy-ethoxy-methyl (MEM). The removal of the hydroxy protecting groups can be effected in a manner known per se for the removal of such groups. For instance, silylether can be removed by treatment with fluoride reagents, such as hydrogen fluoride or tetrabutyl ammonium fluoride in tetrahydrofuran, THP-group by reacting with acid, e. g. pyridinium p-toluenesulfonate in MeOH.

It should be noted that although the intermediates shown in the synthesis of compound of formula I have hydroxy groups typically protected as silylethers, the scope of the invention includes the use of alternative hydroxyl protecting groups known in the art as described in T. W. Greene & P. G. M. Wuts, “Protective Groups in Organic Synthesis,” Wiley, New York (1999) and J. F. McOmie, “Protective Groups in Organic Chemistry,” Plenum Press, London (1973), together with alternative methods for deprotection.

The term “acid protecting group” used herein relates to protecting groups known in the art such as for example 2-trimethylsilyl-ethyl or 2,2,2-trichloroethyl.

In the structural formulae presented herein a broken bond ( [symbol] ) denotes that the substituent is below the plane of the paper and a wedged bond ( [symbol] ) denotes that the substituent is above the plane of the paper, whereas a ( [symbol] )-bond denotes that the substituent is either above or below the plane.

Preferred compound are compounds having the natural configuration at C20.

Preferred compounds of formula I are compounds wherein R⁴ is hydroxy and at least one of Y and Z is hydroxy. Especially preferred are compounds wherein Y and Z are both hydroxy.

Further preferred compounds of formula I are the compounds, wherein A is a group —O(CH₂)₃—, especially the following compound:

(1R,3R)-5-[(E)—(R)-7-(4-ethyl-4-hydroxy-hexyloxy)-oct-2-enylidene]-cyclohexane 1,3-diol.

Further preferred compounds of formula I are the compounds wherein A is a group —CH₂NHCOCH₂ or —CH₂NHCO—, especially the following compounds or mixtures:

N-[(6E,8Z)—(S)-8-[(3S,5R)-3,5-dihydroxy-2-methylene-cyclohexylidene]-2-methyl-oct-6-enyl]-isobutyramide;

mixture of (R)— and (S)—N-[(6E,8Z)—(R)-8-[(3S,5R)-3,5-dihydroxy-2-methylene-cyclohexylidene]-2-methyl-oct-6-enyl]-4,4,4-trifluoro-3-hydroxy-3-methyl-butyramide; and

mixture of (R)— and (S)—N-[(6E,8Z)—(S)-8-[(3S,5R)-3,5-dihydroxy-2-methylene-cyclohexylidene]-2-methyl-oct-6-enyl]-4,4,4-trifluoro-3-hydroxy-3-methyl-butyramide.

A further preferred embodiment of the invention are compounds of formula I wherein A is a group —(CH₂)₂-(1,2-C₆H₄)—, especially preferred compounds are

(Z)-(1R,3S)-5-[(E)—(R)-9-[2-(1-hydroxy-1-methyl-ethyl)-phenyl]-7-methyl-non-2-enylidene]-4-methylene-cyclohexane-1,3-diol; and

(1R,3R)-5-[(E)—(R)-9-[2-(1-hydroxy-1-methyl-ethyl)-phenyl]-7-methyl-non-2-enylidene]-cyclohexane-1,3-diol.

A further preferred embodiment of the invention are compounds of formula I wherein A is a group —C≡C-(1,2-C₆H₄)—, especially preferred is the compound (Z)-(1R,3S)-5-[(E)—(R)-9-[2-(1-hydroxy-1-methyl-ethyl)-phenyl]-7-methyl-non-2-ene-8-ynylidene]-4-methylene-cyclohexane-1,3-diol.

A further preferred group of compounds are the compounds of formula I wherein A is a group —(CH₂)₂—CO—; especially preferred are the compounds

(E)—(R)-12-[(Z)-(3R,5R)-3,5-dihydroxy-2-methylene-cyclohexylidene]-2-hydroxy-2,6-dimethyl-dodec-10-ene-3-one;

(E)—(R)-12-[(3R,5R)-3,5-dihydroxy-cyclohexylidene)-2-hydroxy-2,6-dimethyl-dodec-10-ene-3-one; and

(E)—(R)-2-hydroxy-12-[(Z)-(S)-5-hydroxy-2-methylene-cyclohexylidene]-2,6-dimethyl-dodec-10-en-3-one.

Further preferred compounds are compounds of formula I wherein A is —CH₂—O—CO— and R², R³ and R⁴ are alkyl; especially preferred is the compound 2,2-dimethyl-propionic acid (2R)-8-((3R,5R)-3,5-dihydroxy-cyclohexylidene)-2-methyl-oct-6-enyl ester.

The compounds of formula I can be obtained by cleavage of the silyl protecting group(s) contained in compounds of formula

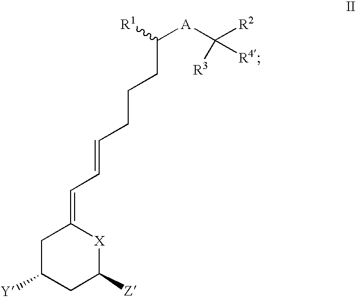

wherein Y′, Z′ are independently of each other hydrogen, fluorine or protected hydroxy groups and R^{4′} is hydrogen, C₁-C₅-alkyl, C₁-C₅-alkoxy or a protected hydroxy group. Preferred hydroxy protecting groups are tert-butyldimethyl-silyl (TBDMS) for the hydroxy groups Y and Z whereas the hydroxy group in R⁴ is preferably protected by trimethyl-silyl [Si(Me)₃].

The cleavage of the hydroxy protecting group(s) can be effected by tetrabutylammonium fluoride (TBAF) in a solvent such as tetrahydrofuran.

The intermediates of formula II, wherein A is —(CH₂)₂-(1,2-C₆H₄)—, —CH═CH—(1,2-C₆H₄)— or —C≡C-(1,2-C₆H₄)— can be prepared according to the reaction Scheme 1. These intermediates are novel and as such are a further object of the present invention.

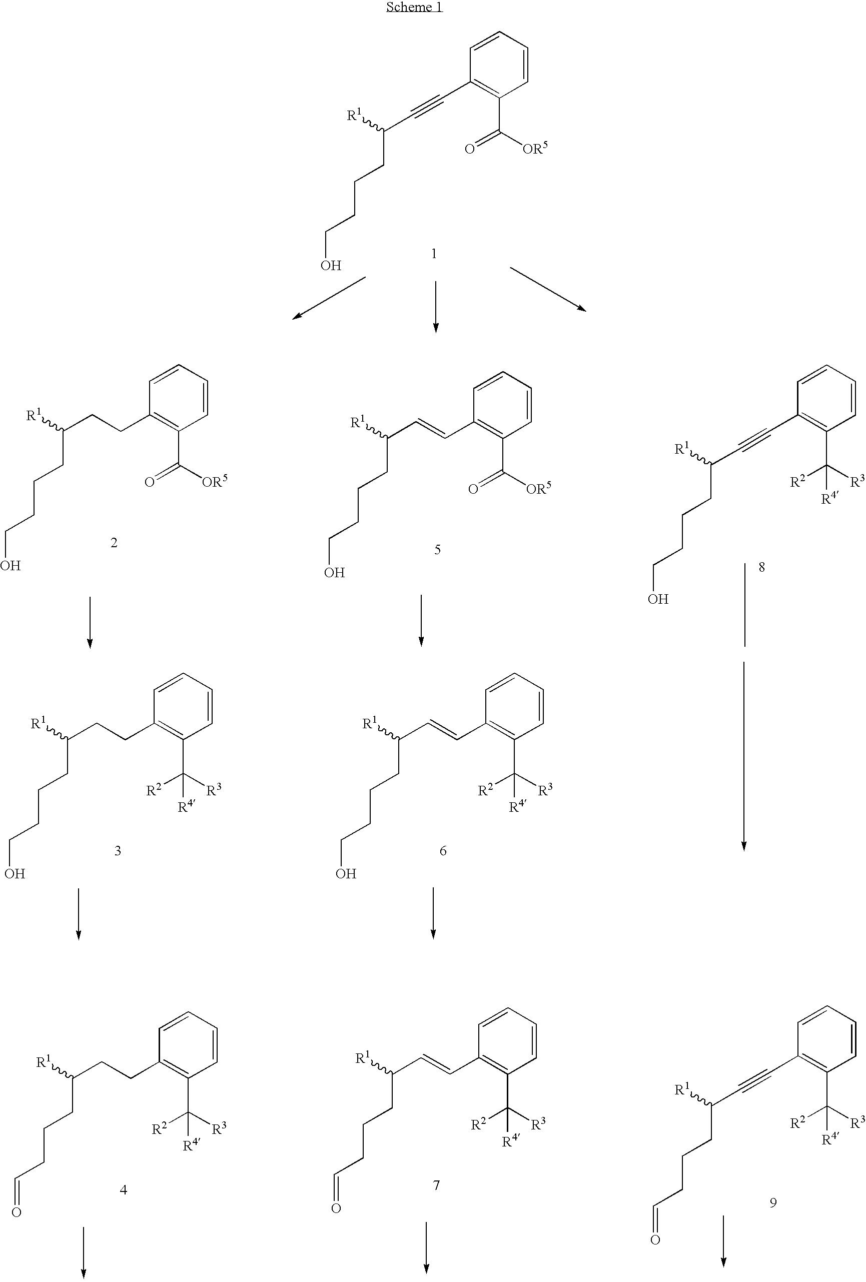

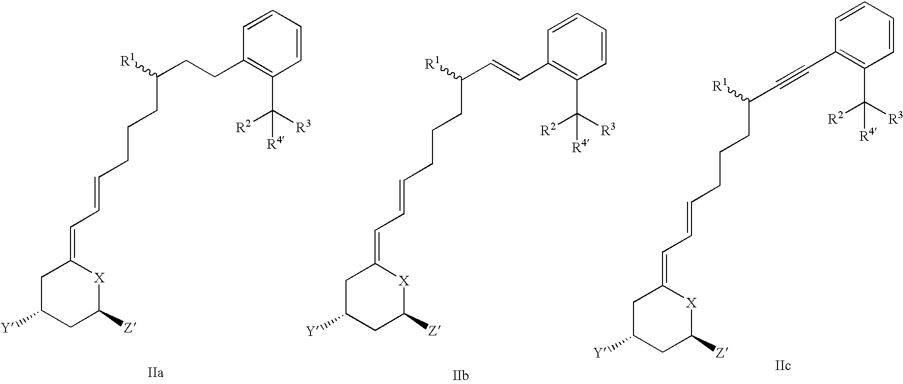

wherein R⁵ is lower alkyl or a carboxylic acid protecting group and R¹, R², R³, R^{4′}, X, Y′ and Z′ are as defined above.

Compounds of formula (1) can be transformed in a manner known per se into the aldehydes (4), (7) or (9). By total or partial reduction of the triple bond of (1) the corresponding alcohols (2) and (5), respectively, are obtained. The ester groups of the compounds (2), (5) and (1), respectively, are reacted via Grignard reaction with alkylmagnesiumhalide to form (3), (6) and (8) (R²═R³) which, after oxidation, form the aldehydes (4), (7) and (9). The aldehydes (4), (7) and (9) are reacted with the corresponding compound of formula III

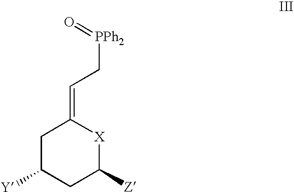

wherein X, Y′ and Z′ are defined as above, in a Wittig reaction to yield the desired intermediates of formula IIa, IIb and IIc.

Compounds of formula III are known from the literature. The symmetrical compounds (Y′=Z′, X is —CH₂—) can be prepared according to Scheme 2

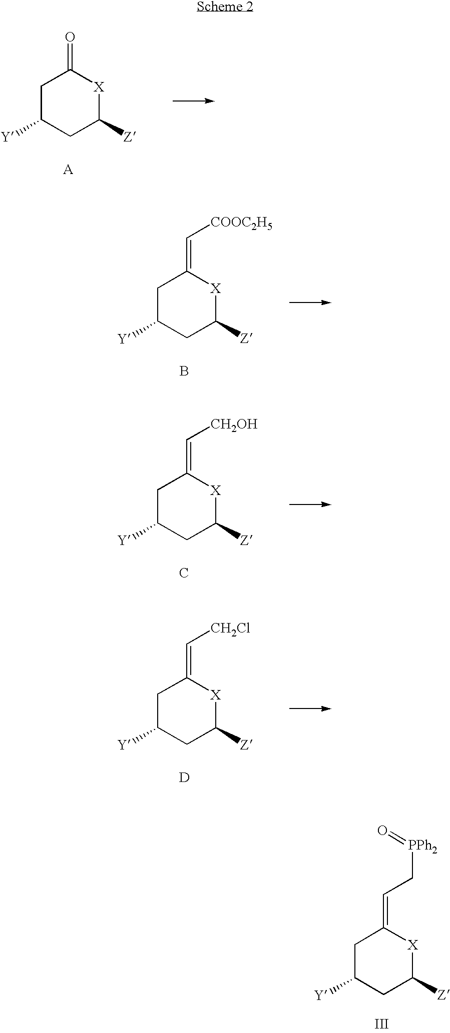

wherein X, Y′ and Z′ are as defined above.

According to Scheme 2, the ketone A is converted by a Peterson reaction into the ester B from which the alcohol C is obtained by reduction. Reaction of the alcohol C with N-chlorosuccinimide in the presence of dimethylsulphide gives the chloride D. Reaction of D with diphenylphosphine-lithium and work-up with 5% H₂O₂ in ethyl acetate produces the phosphine oxide of formula III. The corresponding compound of formula III, wherein Y′ or Z′ is fluorine and X is >CH₂ can be prepared as described in J. Org. Chem. 1990, 55, 243-247.

Compounds of formula (1) are new and also part of the invention. These compounds (1) can be prepared according to the method depicted in Scheme 3:

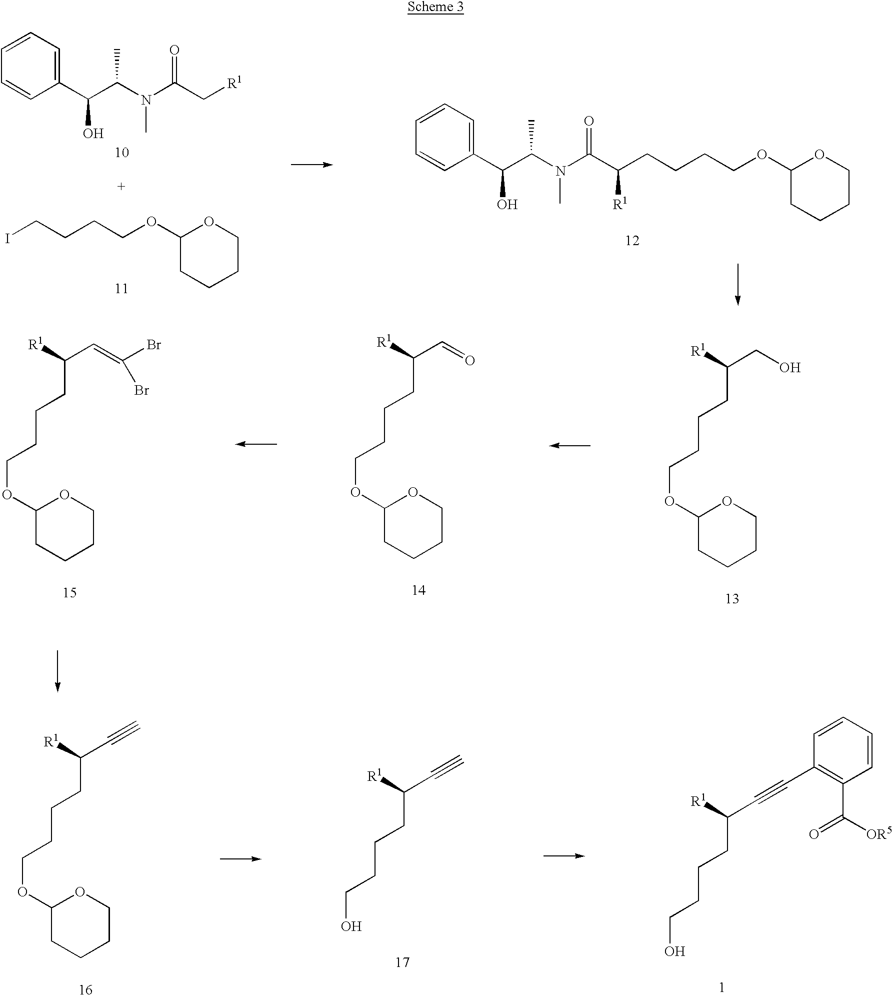

R′ and R′ are as defined above.

An optically active (1S,2S)—N-(2-hydroxy-1-methyl-2-phenyl-ethyl)—N-methyl-alkanoyl amide (10) or its enantiomer is alkylated in the presence of LDA and LiCl with protected 4-iodobutanol ((RS)-2-(4-iodo-butoxy)-tetrahydro-pyran) to form stereoselectively intermediate (12). Reduction leads to alcohol (13), which is oxidized and subjected to a Corey-Fuchs-reaction to afford—via vinyl-dibromide (15)—the terminal acetylene derivative (16). Deprotection and Pd0-catalyzed Sonogashira—coupling delivers finally the target compound (1) in the desired configuration. How is the natural isomer prepared.

The preparation of compounds of formula I, wherein A is a group —O—(CH₂)₃— is depicted in Scheme 4

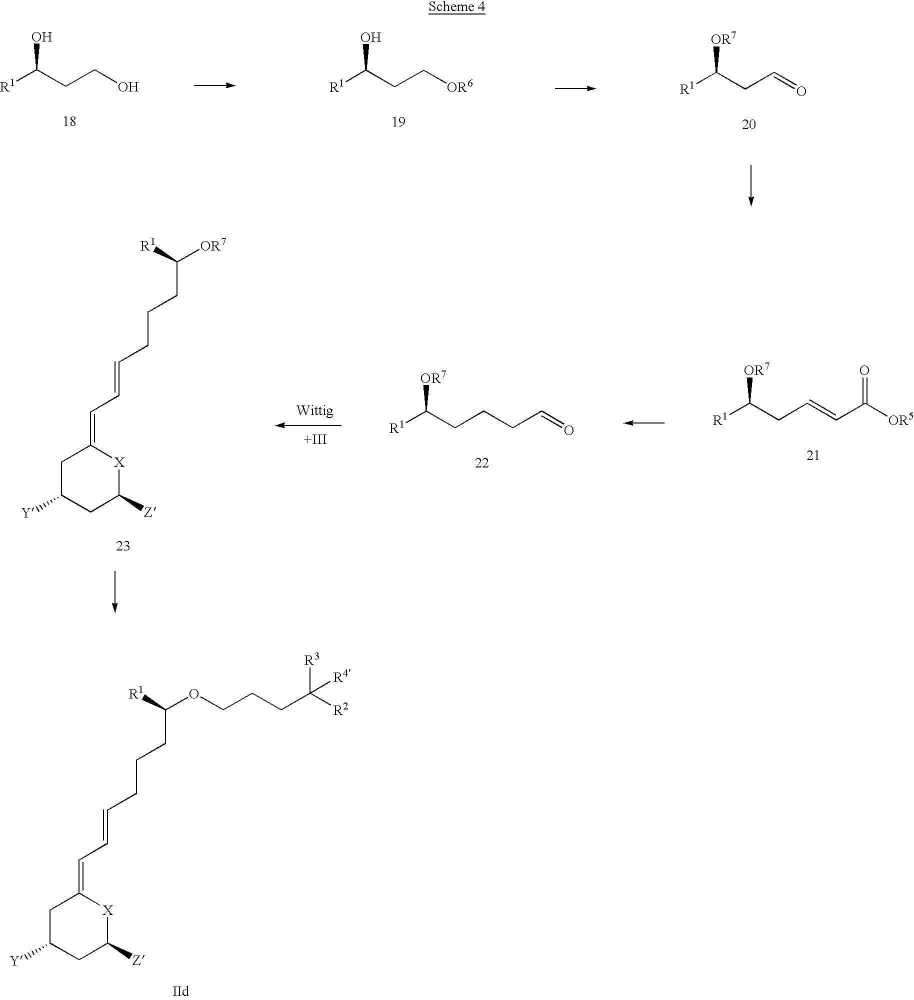

R¹, R^{4′}, X, Y′ and Z′ are as defined above, R⁶ and R⁷ represent hydroxy protecting groups.

The reactions used for the preparation of the intermediates of formula IId are standard reactions used by the person skilled in the art. The diol (18) is reacted with an acid chloride, for example with pivaloyl chloride, to form selectively the corresponding mono ester (19). The remaining hydroxy group is protected with a hydroxy protecting group such as tetrahydropyranyl and the ester cleaved before oxidation of the primary hydroxy group to the corresponding aldehyde (20) with oxalyl chloride/DMSO and triethylamine. The aldehyde is reacted with a stabilized Wittig-reagent to yield the corresponding unsaturated ester (21). The ester is reduced with diisopropyl-aluminum hydride to the corresponding unsaturated alcohol, the double bond in position 2 is hydrogenated in presence of palladium on carbon and the resulting saturated monoprotected alcohol subsequently oxidized with oxalylchloride/DMSO and triethylamine to the aldehyde (22). This aldehyde (22) is then reacted with the corresponding compound of formula III in a Wittig reaction to yield the desired intermediates of formula IId.

The reaction starting with the enantiomer of (18) will yield the corresponding compound of formula IId.

A method for the preparation of compounds of formula I wherein A is a group —CH₂—NH—CO—CH₂— or —CH₂—NH—CO— is depicted in Scheme 5.

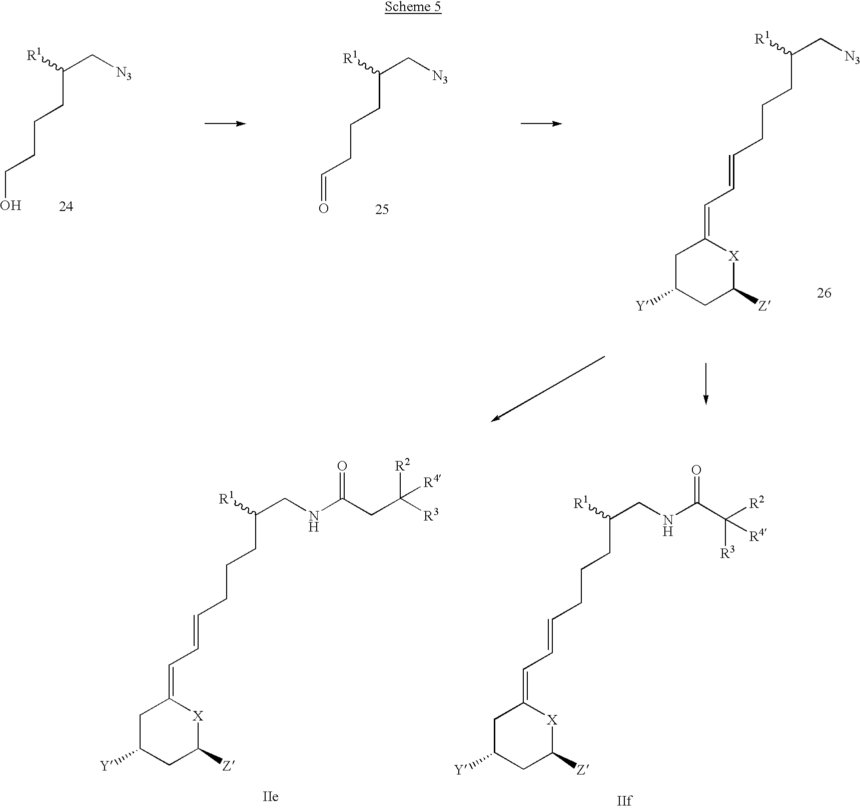

wherein R¹, R², R³, R^{4′}, X, Y′ and Z′ are as defined above.

Compounds of formula IIe and IIf can be prepared starting from an azido aldehyde (25) which can be prepared as desribed in example 3.1.a). The azido aldehyde (25) is then reacted with a compound of formula III in a Wittig reaction to form the intermediate azide (26). The azide is then reduced with triphenylphosphine and water and the resulting primary amine is reacted with an appropriate acid in the presence of dimethylamino-pyridine, triethylamine and dicydohexyl-carbodiimide to form the desired amide of formula IIe or IIf, respectively.

Compounds of formula I wherein A is a group —CH₂—O—CO— can be prepared as depicted in Scheme 6 starting with the monoprotected 1,6-diol (13) in a manner known in the art.

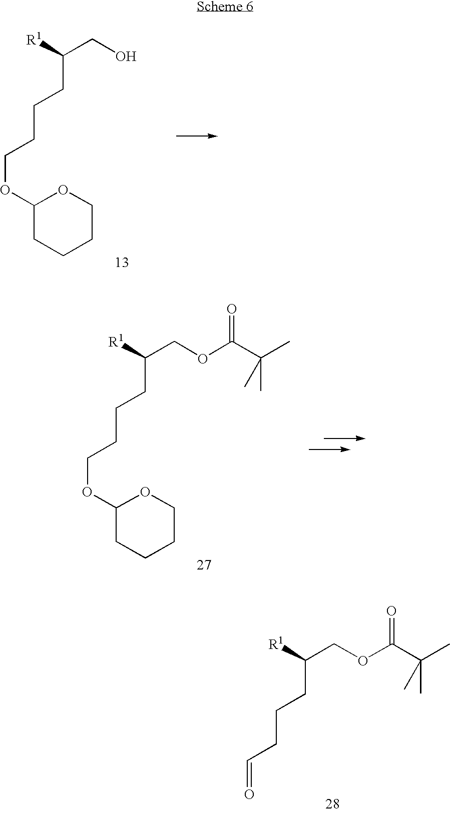

A method for the preparation of compounds of formula I wherein A is a group —(CH₂)₂CO— is depicted in Scheme 7.

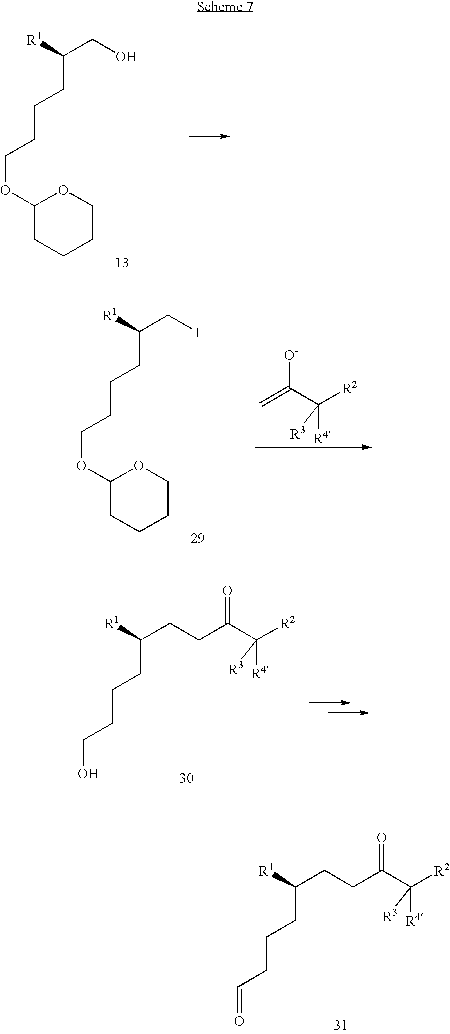

The symbols used in Schemata 6 and 7 are as defined above.

The following examples shall further illustrate the invention, however, without limiting its scope.

### EXAMPLE 1

1.1. Preparation of (1R,3R)-5-[(E)—(R)-7-(4-ethyl-4-hydroxy-hexyloxy)-oct-2-enylidene]-cyclohexane-1,3-diol

a) Preparation of 2,2-dimethyl-propionic acid (R)-3-hydroxy-butyl ester

(R)-1,3-butanediol (5 ml; 55.5 mmol) is dissolved in pyridine (30 ml) and dimethylaminopyridine (678 mg, 5.55 mmol; 0.1 equivalent) is added. The reaction is cooled to 0° C. and pivaloylchloride (8.2 ml; 66.6 mmol; 1.2 equivalent) is added slowly. The mixture is stirred for half an hour at 0° C. and then half an hour at room temperature. The reaction mixture is poured onto a chilled solution of 25% aqueous HCl, extracted twice with ether, the organic phase is washed with brine, dried over sodium sulfate and the solvent is removed. The crude mixture is chromatographed on silica gel (ethyl acetate/hexane 3/7) and 6.95 g (72%) of 2-dimethyl-propionic acid (R)-3-hydroxy-butyl ester is obtained as yellowish oil.

IR (cm⁻¹): 3438; 2971; 2935; 2913; 2875; 1729; 1712; 1482; 1462; 1399; 1368; 1288; 1166; 1141; 1116; 1042. GC-MS: M=174

b) Preparation of 1:1 mixture of (R)—[(R)— and —[(S)-tetrahydro-pyran-2-yloxy]-butyraldehyde

2,2-dimethyl-propionic acid (R)-3-hydroxy-butyl ester (1.00 g; 5.74 mmol) is dissolved in dihydropyran (5 ml), treated with camphor sulfonic acid (CSA) (30 mg), and kept at room temperature for 18 hours. The reaction mixture is poured onto chilled water, extracted twice with ethyl acetate, the organic phase is washed with brine, dried over sodium sulfate and the solvent is removed. The crude mixture is chromatographed on silica gel (ethyl acetate/hexane 1/9) and one obtaines 1.795 g (100%) of the diprotected alcohol. This intermediate (1.78 g; 5.74 mmol) is dissolved in toluene (50 ml) and cooled to −78° C. Diisopropylaluminum hydride (Dibal) (14.3 ml of a 1.2 Mol solution) is added slowly. The mixture is stirred half an hour at −78° C. and is then allowed to warm-up to room temperature. Then methanol (30 ml) is added to the reaction mixture followed by KNa-tartrate (40 ml of a 2N solution); the reaction is stirred till the phase separation is complete. The reaction mixture is poured onto chilled water, extracted twice with ethyl acetate, the organic phase is washed with brine, dried over sodium sulfate and the solvent is removed. The crude mixture is chromatographed on silica gel (isopropanol/hexane 1/9) and 1.00 g (100%) of the monoprotected alcohol is obtained.

Dimethylsulfoxide (DMSO) (0.81 ml; 11.36 mmol; 4.4 equivalents) is dissolved in dichloromethane (15 ml) and cooled to −78° C., followed by slow addition of oxalylchloride (0.80 ml; 9.30 mmol; 3.6 equivalents). The intermediate monoprotected alcohol (0.45 g; 2.58 mmol), dissolved in dichloromethane (8 ml), is then slowly added. The mixture is stirred half an hour at −78° C., then triethylamine (5.04 ml; 36.2 mmol; 14 equivalents) is added, and stirring continued for another 30 minutes, before allowing to warm-up to room temperature. The reaction mixture is then poured onto brine, extracted twice with ethyl acetate, the organic phase is washed with brine, dried over sodium sulfate and the solvents are removed. The crude mixture is chromatographed on silica gel (ethyl acetate/hexane 2/8) and 368 mg (83%) of (R)-(tetrahydro-pyran-2-yloxy)-butyraldehyde is obtained as yellow oil.

IR (cm⁻¹): 2943; 1726; 1138; 1120; 1077; 1034; 1023; 999. GC-MS: M=171

c) Preparation of (E)—(R)-(tetrahydro-pyran-2-yloxy)-hex-2-enoic acid ethyl ester (R)-(tetrahydro-pyran-2-yloxy)-butyraldehyde (365 mg; 2.12 mmol) is dissolved in toluene (25 ml) and ethoxycarbonylmethylen-triphenylphosphoran (1.77 g; 5.09 mmol; 2.4 equivalents) is added and the reaction is stirred at 80° C. for 3 hours. The reaction mixture is poured onto chilled water, extracted twice with ethy lacetate, the organic phase is washed with brine, dried over sodium sulfate and the solvent is removed. The crude mixture is chromatographed on silica gel (ethyl acetate/hexane 15/85) and 500 mg (97%) of E)—(R)-(tetrahydro-pyran-2-yloxy)-hex-2-enoic acid ethyl ester is obtained as yellowish oil.

IR (cm⁻¹): 2942; 1721; 1662; 1266; 1177; 1134; 1119; 1077; 1034; 1023; 994. MS: (M+H):243

d) Preparation of (E)—(R)-(tetrahydro-pyran-2-yloxy)-hex-2-en-1-ol (E)—(R)-(tetrahydro-pyran-2-yloxy)-hex-2-enoic acid ethyl ester (970 mg; 3.97 mmol) is dissolved in toluene (35 ml) and cooled to −78° C. Diisopropyl-aluminum hydride (Dibal) (9.93 ml of a 1.2 Mol solution) is added slowly. The mixture is stirred half an hour at −78° C. and is then allowed to warm-up to room temperature. Then methanol (30 ml) is added to the reaction mixture followed by KNa-tartrate (40 ml of a 2N solution); the reaction is stirred till the phase separation is complete. The reaction mixture is poured onto a chilled water, extracted twice with ethyl acetate, the organic phase is washed with brine, dried over sodium sulfate and the solvent is removed. The crude mixture is chromatographed on silica gel (ethyl acetate/hexane 35/65) to give 702 mg (88%) of(E)—(R)-(tetrahydro-pyran-2-yloxy)-hex-2-en-1-ol as colorless oil.

IR (cm⁻¹): 3420; 1126; 1082; 1028; 991; 952; 910; 878; 720. MS: (M+H):201

e) Preparation of (R)-(tetrahydro-pyran-2-yloxy)-hexanal

((E)—(R)-(tetrahydro-pyran-2-yloxy)-hex-2-en-1-ol (1662 mg; 8.30 mmol) is dissolved in ethanol (40 ml) and palladium on carbon (10%, 80 mg) is added. The reaction mixture is kept under 1 atmosphere of hydrogen over night. The mixture is filtered over Decalite, and the solvent is removed. The crude mixture is chromatographed on silica gel (ethyl acetate/hexane 35/65) and 467 mg (28%) of (R)-(tetrahydro-pyran-2-yloxy)-hexanol is obtained along with 427 mg (25%) of starting material.

Dimethylsulfoxide (0.70 ml; 10.31 mmol; 3 equivalents) is dissolved in dichloromethane (16 ml) and cooled to −78° C., followed by slow addition of oxalylchloride (0.74 ml; 5.56 mmol; 2.5 equivalents). The above-prepared intermediate (0.695 g; 3.44 mmol) is dissolved in dichloromethane (8 ml) and then added slowly via canula. The mixture is stirred for half an hour at −78° C. and, after addition of triethylamine (5.27 ml; 37.8 mmol; 11 equivalents), is allowed to warm-up to room temperature after one additional hour at −78° C. The reaction mixture is poured onto brine, extracted twice with ethyl acetate, the organic phase is washed with brine, dried over sodium sulfate and the solvent is removed. The crude mixture is chromatographed on silica gel (ethyl acetate/hexane 2/8) and 459 mg (67%) of (R)-(tetrahydro-pyran-2-yloxy)-hexanal is obtained as yellow oil.

IR (cm^{31 1}): 2948; 1730; 1142; 1128; 1086; 1030; 1000: 880; 822. MS: (M—CH₃COH): 156

f) Preparation of (E)—(R)-8-[(3R,5R)-3,5-Bis-(tert-butyl-dimethyl-silyloxy)-cyclohexylidene]-2-(tetrahydro-pyran-2-yloxy)-oct-6-ene

(3R,5R)-[2-[3,5-Bis-(tert-butyl-dimethyl-silyloxy)-cyclohexylidene]-ethyl]-diphenyl-phosphine oxide (1140 mg; 2.0 mmol) is dissolved in tetrahydrofuran (8 ml) and cooled down to −78° C. n-BuLi (1.25 ml of a 1.5 M solution; 2 equivalents) is then added slowly and the dark red solution stirred for half an hour. 1:1 Mixture of (R)-5-[(R)— and —[(S)-tetrahydro-pyran-2-yloxy]-hexanal (200 mg; 1 mmol), dissolved in tetrahydrofuran (4 ml), is then added via canula. After one hour stirring at −78° C., the mixture is stirred one additional hour at 0° C. The reaction mixture is poured onto chilled saturated ammonium chloride solution, extracted twice with ethyl acetate, the organic phase is washed with brine, dried over sodium sulfate and the solvent is removed. The crude mixture is chromatographed on silica gel (ethyl acetate/hexane 4/96) and 185 mg (34%) of E)—(R)-8-[(3R,5R)-3,5-bis-(tert-butyl-dimethyl-silyloxy)-cyclohexylidene]-2-(tetrahydro-pyran-2-yloxy)-oct-6-ene is obtained as yellow oil.

IR (cm⁻¹): 2953; 29296; 2856; 1472; 1463; 1255; 1127; 1086; 1051; 1023; 1006; 962; 836; 775. MS: (M): 552

g) Preparation of (E)—(R)-8-[(3R,5R)-3,5-Bis-(tert-butyl-dimethyl-silyloxy)-cyclohexylidene]-2-(tetrahydro-pyran-2-yloxy)-oct-6-ene

(E)—(R)-8-[(3R,5R)-3,5-Bis-(tert-butyl-dimethyl-silyloxy)-cyclohexylidene]-oct-6-en-2-ol (85 mg; 0.153 mmol) is dissolved in dichloromethane (4 ml). The reaction mixture is cooled to −78° C. and dimethylaluminum chloride (0.3 ml of a 1 M solution; 2 equivalents) is added slowly. After 15 minutes stirring at −78° C. the mixture is kept for two hours at room temperature. The reaction mixture is poured onto chilled KNa-tartrate solution, extracted twice with ethyl acetate, the organic phase is washed with brine, dried over sodium sulfate and the solvent is removed. 71 mg (100%) (E)—(R)-8-[(3R,5R)-3,5-Bis-(tert-butyl-dimethyl-silyloxy)-cyclohexylidene]-2-(tetrahydro-pyran-2-yloxy)-oct-6-ene is obtained as yellow oil.

IR (cm⁻¹): 3370; 2955; 2929; 2886: 2857; 1477; 1255; 1086; 1052; 962; 836; 775. MS: (M): 468

h) Preparation of (E)—(R)-14-[(3R,5R)-3,5-Bis-(tert-butyl-dimethyl-silyloxy)-cyclohexylidene]-3-ethyl-8-methyl-7-oxa-3-(trimethyl-silyloxy)-tetradeca-12-ene

(E)—(R)-8-[(3R,5R)-3,5-Bis-(tert-butyl-dimethyl-silyloxy)-cyclohexylidene]-2-(tetrahydro-pyran-2-yloxy)-oct-6-ene (83 mg; 0.177 mmol) is dissolved in tetrahydrofuran (5 ml). The reaction mixture is cooled to 0° C. and potassium hydride (71 mg; 2 equivalents) is added slowly. After 15 minutes stirring at 0° C. n-tetrabutyl-ammonium iodide (6.5 mg; 0.1 equivalent), crown ether 18-C-6 (4.7 mg; 0.1 equivalent), and 6-bromo-3-ethyl-3-(trimethyl-silyloxy)-hexane (100 mg, 0.354 mmol; 2 equivalents) are added. The mixture is then stirred for 3 hours at room temperature. The reaction mixture is poured onto chilled water, extracted twice with ethyl acetate; the organic phase is washed with brine, dried over sodium sulfate and the solvent is removed. The crude mixture is chromatographed on silica gel (ethyl acetate/hexane 2/98) and 60 mg (51%) of (E)—(R)-14-[(3R,5R)-3,5-bis-(tert-butyl-dimethyl-silyloxy)-cyclohexylidene]-3-ethyl-8-methyl-7-oxa-3-(trimethyl-silyloxy)-tetradeca-12-ene is obtained as yellow oil.

MS: (M): 668

NMR (CDCl₃, J in Hz, 250 MHz): 6.62 (dd; J=15.0; 10.8; 1H), 5.83 (d; J=10.8; 1H); 5.57 (dt; J=15.0; 6.6; 1H); 4.05 (m; 2H); 3.50-3.24 (m; 3H); 2.28 (m; 3H), 1.70 (m; 2H); 1.54-1.28 (m; 12H), 1.11 (d; J=6.0; 3H); 0.85 (s; 18H); 0.85 (t; J=7.3; 6H); 0.10-0.00 (m; 15H).

i) Preparation of (E)—(R)-14-[(3R,5R)-3,5-dihydroxy-cyclohexylidene]-3-ethyl-8-methyl-7-oxa-tetradeca-12-en-3-ol

(E)—(R)-14-[(3R,5R)-3,5-bis-(tert-butyl-dimethyl-silyloxy)-cyclohexylidene]-3-ethyl-8-methyl-7-oxa-3-(trimethyl-silyloxy)-tetradeca-12-ene (56 mg; 0.084 mmol) is dissolved in tetrahydrofuran (3.5 ml). Tetrabutylammonium fluoride (0.84 ml of a 1M solution; 10 equivalents) is added and the mixture is allowed to react at room temperature for 20 hours. The reaction mixture is poured onto chilled water, extracted twice with ethyl acetate, the organic phase is washed with brine, dried over sodium sulfate and the solvent is removed. The crude mixture is chromatographed on silica gel (isopropanol/hexane 2/8) and 23 mg (75%) of (E)—(R)-14-[(3R,5R)-3,5-dihydroxy-cyclohexylidene]-3-ethyl-8-methyl-7-oxa-tetradeca-12-en-3-ol is obtained as colorless oil.

MS: (M+H): 369 NMR (CDCl₃, J in Hz, 250 MHz): 6.28 (dd; J=14.; 10.0; 1H), 5.99 (d; J=10.0; 1H); 5.67 (dt; J=14.8; 6.6; 1H); 4.09 (m; 2H); 3.56-3.28 (m; 3H); 2.60 (dd; J=13.2; 4.0; 1H), 2.46 (dd; J=13.2; 4.0; 1H), 2.29 (dd; J=13.2; 7.6; 1H), 2.10 (m; 3H), 1.84 (m; 2H); 1.72-1.17 (m; 15H), 1.09 (d; J=6.1; 3H); 0.85 (t; J=7.5; 6H.

### EXAMPLE 2

2.1. Preparation of (Z)-(1R,3S)-5-[(E)—(R)-9-[2-(1-hydroxy-1-methyl-ethyl)-phenyl]-7-methyl-non-2-enylidene]-4-methylene-cyclohexane-1,3-diol

a) Preparation of 2-(4-iodo-butoxy)-tetrahydro-pyran from 1,4-butanediol

To 1,4-butanediol (50 ml; 560 mmol) is successively added dichloromethane (10 ml), dihydropyran (25.5 ml; 280 mmol), and finally p-toluenesulfonic add (200 mg). The mixture is allowed to react over night. The reaction mixture is poured onto chilled water, extracted twice with ethyl acetate, the organic phase is washed with brine, dried over sodium sulfate and the solvents are removed. The crude mixture is chromatographed on silica gel (ethyl acetate/hexane 2/8 and then ethyl acetate/hexane 4/6) to yield 34.8 g (71%) of 2-(4-hydroxy-butoxy)-tetrahydro-pyran as colorless oil.

The mono-protected butanediol (5.0 g; 28.7 mmol) is dissolved in ether/acetonitrile (3:1,200 ml). Triphenylphosphine (9.41 g; 35.87 mmol; 1.25 equivalents) and imidazole (3.90 mmol; 57.5 mmol; 2 equivalents) are added. Then iodine (9.1 g; 35.87 mmol; 1.25 equivalents) is added in several portions at room temperature. A persistent orange coloration of the reaction mixture and an abundant yellow precipitate indicate the end of the reaction. The precipitate is filtered off and the solvents are removed. The crude mixture is chromatographed on silica gel (ethylacetate/hexane 1/9) and 7.15 g (88%) of 2-(4-iodo-butoxy)-tetrahydro-pyran are obtained as a pale reddish oil.

IR (cm⁻¹): 2939; 2866; 1452; 1441; 1365; 1352; 1261; 1226; 1201; 1184; 1134; 1190; 1066; 1034; 988; 904; 869. MS: (M−H): 283

b) Preparation of (1S,2S)—N-(2-hydroxy-1-methyl)-2-phenyl-ethyl)-N-methyl-propionamide

(1S,2S)—N-(2-Hydroxy-1-methyl-2-phenyl-ethyl)-N-methyl-propionamide is prepared according to the method described by Myers (Myers et al; J Am Chem Soc; 1994; 116; 9361)

c) Preparation of (R)-2-methyl-6-[tetrahydro-pyran-2-yloxy)]-hexanoic acid (1S,2S)—N-(2-hydroxy-1-methyl)-2-phenyl-ethyl)-N-methyl-amide

Diisopropylamine (7.2 ml; 50.83 mmol; 2.25 equivalents) is diluted with anhydrous tetrahydrofuran (70 ml). At 0° C. BuLi (31.8 ml of a 1.6 M solution; 50.83 mmol; 2.25 equivalents) is added slowly and the mixture is stirred for 15 minutes. Lithium chloride (5.75 g; 135.6 mmol; 6 equivalents) is added and the mixture is cooled to −78° C. (1S,2S)—N-(2-Hydroxy-1-methyl)-2-phenyl-ethyl)-N-methyl-propionamide (6.0 g, 22.6 mmol), dissolved in anhydrous tetrahydrofuran (70 ml) is then added slowly and the mixture is stirred for one hour at −78° C. and for 15 minutes at ambient temperature. The mixture is again cooled to −78° C. and the above prepared iodide 2-(4-iodo-butoxy)-tetrahydro-pyran (7.06 g; 24.86 mmol; 1.1 equivalent) is added slowly. The mixture is warmed to 0° C. and stirring is continued for one hour to complete the alkylation. The reaction mixture is poured onto chilled brine, extracted twice with ethyl acetate, the organic phase is washed with brine, dried over sodium sulfate and the solvent is removed. The crude mixture is chromatographed on silica gel (ethyl acetate/hexane 8/92) and 8.45 g (99%) of (R)-2-methyl-6-[tetrahydro-pyran-2-yloxy)]-hexanoic acid (1S,2S)—N-(2-hydroxy-1-methyl)-2-phenyl-ethyl)-N-methyl-amide is obtained as yellow oil.

IR (cm−1): 3393; 2940; 2869; 1620; 1453; 1409; 1375; 1136; 1120; 1078; 1031; 767; 702. MS: (M+H+): 378

d) Preparation of (R)-2-methyl-6-[tetrahydro-pyran-2-yloxy)]-hexan-1-ol

Ammonia-borane complex (BH₃—NH₃) (3.05 g; 89 mmol; 4 equivalents) is dissolved in anhydrous tetrahydrofuran (125 ml). At 0° C. nBuLi (54 ml of a 1.6 M solution; 87 mmol; 4 equivalents) is added slowly and the mixture is stirred for 15 minutes. Then (R)-2-methyl-6-[tetrahydro-pyran-2-yloxy)]-hexanoic acid (1S,2S)—N-(2-hydroxy-1-methyl)-2-phenyl-ethyl)-N-methyl-amide (8.2 g; 21.72 mmol), dissolved in tetrahydrofuran (25 ml), is added slowly and allowed to react over night. The reaction mixture is poured onto chilled brine, extracted twice with ethyl acetate, the organic phase is washed with brine, dried over sodium sulfate and the solvent is removed. The crude mixture is chromatographed on silica gel (ethyl acetate/hexane 3/7) and 4.41 g (94%) of (R)-2-methyl-6-[tetrahydro-pyran-2-yloxy)]-hexan-1-ol is obtained as colorless oil.

IR (cm−1): 3422; 2938; 2870; 1465; 1454; 1353; 1323; 1201; 1137; 1122; 1079; 1033; 988; 911; 874; 820. MS: (M—C₄H₉O ): 143

e) Preparation of (R)-2-methyl-6-[tetrahydro-pyran-2-yloxy)]-hexanal

(R)-2-methyl-6-[tetrahydro-pyran-2-yloxy)]-hexane-1-ol (3.00 g; 13.87 mmol) is dissolved in dichloromethane (50 ml). N-methyl morpholine N-oxide (NMO) (2.44 g; 20.8 mmol; 1.5 equivalent) and crushed molecular sieves (3 g) are added and stirring continued for one hour. The mixture is then cooled to −78° C. and tetrapropyl ammonium perruthenate (TPAP) (70 mg; 0.2 mmol; 1.5 mol %) is added and the mixture is allowed to reach slowly room temperature. Completion of the reaction is monitored by thin layer chromatography.

The reaction mixture is directly poured onto a silica gel column, and eluted with ethyl acetate/hexane (15/85) to give 2.18 g (73%) of (R)-2-methyl-6-[tetrahydro-pyran-2-yloxy)]-hexanal as colorless oil.

IR (cm⁻¹):2941; 2869; 1739; 1455; 1442; 1373; 1353; 1241; 1201; 1132; 1079; 1034; 988; 906; 869; 819. MS: (M—C₅H₉O): 129

f) Preparation of 2-[(R)-7,7-dibromo-5-methyl-hept-6-enyloxy]-tetrahydro-pyran

Tetrabromomethane (7.4 g; 44.6 mmol; 4.4 equivalents) is dissolved in absolute dichloromethane (150 ml) and triphenylphosphine (7.4 g; 22.3 mmol; 2.2 equivalents) as dichloromethane-solution (50 ml) is added slowly at 0° C. The reaction mixture is then cooled to −10° C. and (R)-2-methyl-6-[tetrahydro-pyran-2-yloxy)]-hexanal is added carefully. After 30 minutes the reaction is gone to completion. The reaction mixture is poured onto chilled saturated bicarbonate solution, extracted twice with ethyl acetate, the organic phase is washed with brine, dried over sodium sulfate and the solvent is removed. The crude mixture is chromatographed on silica gel (ethyl acetate/hexane 1/9) to produce 1.37 g (37%) of 2-[(R)-7,7-dibromo-5-methyl-hept-6-enyloxy]-tetrahydro-pyran as colorless oil.

IR (cm⁻¹): 2938; 2868; 1622; 1453; 1137; 1123; 1033; 781. MS: (M−H ): 371

g) Preparation of 2-[(R)-5-methyl-hept-6-ynyloxy]-tetrahydro-pyran

2-[(R)-7,7-dibromo-5-methyl-hept-6-enyloxy]-tetrahydro-pyran (1.40 g; 3.78 mmol) is dissolved in anhydrous tetrahydrofuran (20 ml). At −78° C. nBuLi (5.4 ml of a 1.6 M solution; 8.7 mmol; 2.3 equivalents) is added slowly and the mixture is stirred for 15 minutes. The mixture is allowed to reach room temperature, poured onto chilled water, extracted twice with ethyl acetate, the organic phase is washed with brine, dried over sodium sulfate, and the solvent is removed. The crude mixture is chromatographed on silica gel (ethyl acetate/hexane 1/9) and 470 mg (59%) of 2-[(R)-5-methyl-hept-6-ynyloxy]-tetrahydro-pyran is obtained as colorless oil.

IR (cm⁻¹): 3309; 2939; 2871; 1454; 1201; 1137; 1121; 1079; 1034; 996; 913; 877; 723. NMR (CDCl₃, J in Hz, 250 MHz): 4.58 (m, 1H); 3.89 (m, 1H); 3.77 (m, 1H); 3.50 (m, 1H); 3.39 (m, 1H); 2.45 (qm; J=6.8; 1H); 2.03 (d, J=2.4; 1H); 1.92-1.43 (m, 12H), 1.18 (d; J=6.8; 3H).

h) Preparation of (R)-5-methyl-hept-6-yn-1-ol

2-[(R)-5-Methyl-hept-6-ynyloxy]-tetrahydro-pyran (1180 mg; 5.61 mmol) is dissolved in methanol (20 ml). A 25% solution of aqueous HCl (0.8 ml) is added and the mixture is allowed to react over night. The reaction mixture is poured onto chilled water, extracted twice with ethyl acetate, the organic phase is washed with brine, dried over sodium sulfate and the solvent is removed. The crude mixture is chromatographed on silica gel (ethyl acetate/hexane 3/7) to yield 530 mg (75%) of(R)-5-methyl-hept-6-yn-1-ol as colorless oil.

GC-MS: (M ): 126

IR (cm⁻¹): 3303; 2971; 2936; 2864; 2120; 1455; 1382; 1072; 1047.

i) Preparation of (R)-2-(7-hydroxy-3-methyl-hept-1-ynyl) benzoic acid ethyl ester

(R)-5-Methyl-hept-6-yn-1-ol (560 mg; 5.14 mmol) is dissolved in piperidine (10 ml). 2-Iodo-benzoic acid ethyl ester (2.1 g; 7.6 mmol; 1.5 equivalent) is added. The reaction vessel is degassed and flushed with argon.

Bis(triphenylphosphine)palladium dichloride (180 mg; 0.256 mmol; 0.05 equivalent) and copper (I) chloride (48 mg; 0.256 mmol; 0.05 equivalent) is added and the mixture is heated to 60° C. over night. The reaction mixture is poured onto a chilled 25% solution of aqueous HCl, extracted twice with ethyl acetate, the organic phase is washed with brine, dried over sodium sulfate and the solvent is removed. The crude mixture is chromatographed on silica gel (ethyl acetate/hexane 15/85) and 1200 mg (85%) of (R)-2-(7-hydroxy-3-methyl-hept-1-ynyl) benzoic acid ethyl ester is obtained as colorless oil.

MS: (M): 274 IR (cm⁻¹): 3440; 2980; 2960; 2878; 2238; 1727; 1711; 1604; 1562; 1488; 1452; 1290; 1274; 1249; 1137; 1078; 1049; 759; 708.

j) Preparation of (R)-2-(7-hydroxy-3-methyl-heptyl) benzoic acid ethyl ester

(R)-2-(7-Hydroxy-3-methyl-hept-1-ynyl) benzoic acid ethyl ester (600 mg; 2.18 mmol) is dissolved in ethanol (20 ml). Palladium on carbon (10%) (50 mg) is added and the mixture hydrogenated under 1 atmosphere of hydrogen over night. The reaction mixture is filtered over Decalite and the solvent is removed. 460 mg (77%) of (R)-2-(7-hydroxy-3-methyl-heptyl) benzoic acid ethyl ester is obtained as colorless oil.

MS: (M): 278 IR (cm⁻¹): 3367; 2932; 2866; 1720; 1601; 1461; 1448; 1366; 1293; 1257; 1136; 1100; 1073; 1047; 1021; 751; 710.

k) Preparation of (R)-3-methyl 1-[2-(1-hydroxy-1-methyl-ethyl)-phenyl]-heptane-7-ol

(R)-2-(7-Hydroxy-3-methyl-heptyl) benzoic acid ethyl ester (445 mg; 1.6 mmol) is dissolved in anhydrous tetrahydrofuran (10 ml). At 0° C., methylmagnesium chloride (6.4 ml of a 3 M solution; 19.2 mmol; 12 equivalents) is added slowly and the mixture is stirred for two hours. The reaction mixture is carefully poured onto a chilled citric acid solution (1M in water), extracted twice with ethyl acetate, the organic phase is washed with brine, dried over sodium sulfate and the solvent is removed. The crude mixture is chromatographed on silica gel (isopropanol/hexane 1/9) to produce 400 mg (94%) of (R)-3-methyl 1-[2-(-1-hydroxy-1-methyl-ethyl)-phenyl]-heptane-7-ol as colorless oil.

MS: (M—CH₃):249 IR (cm⁻¹): 3362; 2931; 2864; 1608; 1582; 1461; 1443; 1377; 1364; 1050; 962; 872; 760; 749.

1) Preparation of (R)-3-methyl 1-[2-(1-hydroxy-1-methyl-ethyl)-phenyl]-heptane-7-al

(R)-3-Methyl 1-[2-(-1-hydroxy-1-methyl-ethyl)-phenyl]-heptane-7-ol (380 mg; 1.43 mmol) is dissolved in a dichloromethane/dimethylsulfoxide mixture (9:1, 12 ml) and cooled to 0° C. Triethylamine (0.52 ml; 3.7 mmol; 2.6 equivalents) is added followed by pyridine-SO₃ complex (421 mg; 2.65 mmol; 1.85 equivalent). The mixture is stirred for two hours at 0° C. The reaction mixture is then poured onto brine, extracted twice with ethyl acetate, the organic phase is washed with brine, dried over sodium sulfate and the solvent is removed. The crude mixture is chromatographed on silica gel (ethyl acetate/hexane 25/75) and 300 mg (80%) of (R)-3-methyl 1-[2-(-1-hydroxy-1-methyl-ethyl)-phenyl]-heptane-7-al is obtained as colorless oil.

MS: (M—CH₃):247 IR (cm⁻¹): 2952; 2928 2869; 2738; 1722; 1492; 1461; 1443; 1379; 1365; 1139; 1063; 960; 872; 762.

m) Preparation of 2-[2-[(7E,9Z)—(R)-9-[(3S,5R)-3,5-bis-(tert-butyl-dimethyl-silyloxy)-2-methylene-cyclohexylidene]-3-methyl-non-7-enyl]-phenyl]-propan-2-ol

[3S-(3alpha,5beta,Z)]-2-[2-[2-Methylene-3,5-bis-[[(1,1-dimethylethyl)dimethyl-silyl]oxy]cyclo-hexylidene]ethyl]diphenyl phosphine oxide (933 mg; 1.6 mmol; 3 equivalents) is dissolved in anhydrous tetrahydrofuran (3 ml). The solution is cooled to −78° C., nBuLi (1.07 ml of a 1.5 M solution; 1.6 mmol; 3 equivalents) is added and the deep red solution is stirred for 30 minutes. (R)-3-Methyl 1-[2-(1-hydroxy-1-methyl-ethyl)-phenyl]-heptane-7-al (140 mg; 0.53 mmol), dissolved in anhydrous tetrahydrofuran (2 ml), is added, and the mixture allowed to react at −78° C. for 1.5 hours, before the reaction is quenched with methanol (0.5 ml). The reaction mixture is poured onto bicarbonate solution (1 M), extracted twice with ethyl acetate, the organic phase is washed with brine, dried over sodium sulfate and the solvent is removed. The crude product is dissolved in anhydrous tetrahydrofuran (30 ml) and cooled to 0° C. Sodium hydride (150 mg (55%); 3.44 mmol; 6.5 equivalents) is added and the temperature allowed to reach room temperature and kept for two hours to complete the elimination.

The reaction mixture is poured onto a citric acid solution (1M), extracted twice with ethyl acetate, the organic phase is washed with brine, dried over sodium sulfate and the solvent is removed. The crude mixture is chromatographed on silica gel (ethyl acetate/hexane 1/9) and 270 mg (81%) of 2-[2-[(7E,9Z)—(R)-9-[(3S,5R)-3,5-bis-(tert-butyl-dimethyl-silyloxy)-2-methylene-cyclohexylidene]-3-methyl-non-7-enyl]-phenyl]-propane-2-ol is obtained as colorless oil, contaminated with small amounts of the 7Z-isomer.

MS: (M): 626 IR (cm⁻¹): 2955; 2928; 2856; 1472; 1463; 1444; 1377; 1361; 1255; 1086; 1006; 989; 908; 836; 776.

n) Preparation of (Z)-(1R,3S)-5-[(E)—(R)-9-[2-(1-hydroxy-1-methyl-ethyl)-phenyl]-7-methyl-non-2-enylidene]-2-methylene-cyclohexane-1,3-diol

2-[2-[(7E,9Z)—(R)-9-[(3S,5R)-3,5-Bis-(tert-butyl-dimethyl-silyloxy)-2-methylene-cyclohexylidene]-3-methyl-non-7-enyl]-phenyl]-propane-2-ol (260 mg; 0.34 mmol) is dissolved in tetrahydrofuran (5 ml) and tetrabutyl-ammonium fluoride (5 ml of a 1M solution; 5 mmol; 14 equivalents) is added. The mixture is allowed to react over night. The reaction mixture is poured onto brine, extracted twice with ethyl acetate, the organic phase is washed with brine, dried over sodium sulfate, and the solvent is removed. The crude mixture is chromatographed on silica gel (isopropanol/hexane 25/75) to give 87 mg (53%) of (Z)-(1R,3S)-5-[(E)—(R)-9-[2-(1-hydroxy-1-methyl-ethyl)-phenyl]-7-methyl-non-2-enylidene]-2-methylene-cyclohexane-1,3-diol as colorless foam, also contaminated with small amounts of the double bond isomer.

MS: (M): 398 IR (cm⁻¹): 3350; 2938; 2880; 1466; 1450; 1384; 1373; 1152; 1047; 982; 962; 921; 872; 758; 748.

2.2. Preparation of (1R,3R)-5-[(E)—(R)-9-[2-(1-hydroxy-1-methyl-ethyl)-phenyl]-7-methyl-non-2-enylidene]-cyclohexane-1,3-diol

(1R,3R)-5-[(E)—(R)-9-[2-(1-Hydroxy-1-methyl-ethyl)-phenyl]-7-methyl-non-2-enylidene]-cydohexane-1,3-diol is prepared in analogy to example 2.1.

a) 2-[2-[(7E,9Z)—(R)-9-[(3S,5R)-3,5-Bis-(tert-butyl-dimethyl-silyloxy)-cyclohexylidene]-3-methyl-non-7-enyl]-phenyl]-propan-2-ol

MS: (M): 614 IR (cm⁻¹): 2955; 2927; 2856; 1482; 1463; 1361; 1254; 1086; 1052; 1026; 962; 836; 775.

b) (Z)-(1R,3S)-5-[(E)—(R)-9-[2-(1-Hydroxy-1-methyl-ethyl)-phenyl]-7-methyl-non-2-enylidene]-cyclohexane-1,3-diol

MS: (M): 386 IR (cm⁻¹): 3350; 2938; 2880; 1466; 1450; 1384; 1360; 1150; 1046; 961; 872; 822; 758; 748.

2.3. Preparation of (Z)-(1R,3S)-5-[(E)—(R)-9-[2-(1-hydroxy-1-methyl-ethyl)-phenyl]-7-methyl-non-2-en-8-ynylidene]-4-methylene-cyclohexane-1,3-diol

(Z)-(1R,3S)-5-[(E)—(R)-9-[2-(1-Hydroxy-1-methyl-ethyl)-phenyl]-7-methyl-non-2-en-8-ynylidene]-4-methylene-cyclohexane-1,3-diol is prepared in analogy to example 2.1, but omitting the hydrogenation step j).

a) (R)-3-Methyl 1-[2-(-1-hydroxy-1-methyl-ethyl)-phenyl]-hept-6-ynyl-7-ol MS: (M—CH₃):245

IR (cm⁻¹): 3373; 2969; 2934; 2868; 1482; 1440; 1375; 1365; 1175; 1071; 1054; 962; 760.

b) (R)-3-Methyl 1-[2-(-1-hydroxy-1-methyl-ethyl)-phenyl]-hept-6-ynyl-7-al

MS: (M): 259 IR (cm⁻¹): 3465; 2971; 2933; 2871; 1725; 1602; 1459; 1440; 1385; 1365; 1336; 1234; 1173; 1136; 1109; 1046; 958; 763.

c) 2-[2-[(7E,9Z)—(R)-9-[(3S,5R)-3,5-Bis-(tert-butyl-dimethyl-silyloxy)-2-methylene-cyclohexylidene]-3-methyl-non-7-en-1-ynyl]-phenyl]-propane-2-ol

MS: (M): 622 NMR (CDCl₃, J in Hz, 250 MHz): 7.45 (m, 2H); 7.20 (m, 2H); 6.39 (dd; J=15.2; 9.6; 1H); 5.91 (d; J=9.6; 1H); 5.63 (dt, J=15.2; 6.8; 1H); 5.20 (m, 1H); 4.88 (m, 1H); 4.45 (t; J=5.6; 1H); 4.18 (m; 1H); 3.81 (s; 1H); 2.71 (qm; J=6.9; 1H); 2.50-1.00 (m; 12H); 1.70 (s; 6H); 1.27 (d; J=6.9; 3H); 0.87 (s; 18H), 0.06 (s; 6H); 0.05(s; 6H).

d) (Z)-(1R,3S)-5-[(E)—(R)-9-[2-(1-Hydroxy-1-methyl-ethyl)-phenyl]-7-methyl-non-2-en-8-ynylidene]-2-methylene-cyclohexane-1,3-diol

MS: (M): 394 IR (cm−1): 3360; 2980; 2938; 2880; 1445; 1370; 1180; 1048; 974; 953; 912; 757.

### EXAMPLE 3

3.1. Preparation of Mixture of (R)— and (S)—N-[(6E,8Z)—(R)-[(3S,5R)-3,5-dihydroxy-2-methylene-cyclohexylidene]-2-methyl-oct-6-enyl]-4,4,4-trifluoro-3-hydroxy-3-methyl-butyramide

This compound is prepared according to the methods described in Example 2.1; crucial intermediates are obtained as follows:

a) Preparation of (R)-6-azido-5-methyl-hexanal

(R)-2-Methyl-6-[tetrahydro-pyran-2-yloxy]-hexane-1-ol (1.76 g; 8.15 mmol) is dissolved in dichloromethane (20 ml). Dimethylamino-pyridine (2.0 g; 16.3 mmol; 2 equivalent) is added and the solution is cooled to 0° C. Mesylchloride (1.95 ml; 12.23 mmol; 1.5 equivalents) is added and the mixture is allowed to react for two hours.

The reaction mixture is poured onto brine, extracted twice with ethyl acetate. The organic phase is washed with brine, dried over sodium sulfate, and the solvent is removed. The crude residue is dissolved in dimethylsulfoxide (DMSO, 15 ml), sodium azide (1.6 g; 24.45 mmol; 3 equivalents) is added and the reaction allowed to proceed for three hours. The reaction mixture is poured onto brine, extracted twice with ethyl acetate, the organic phase is washed with brine, dried over sodium sulfate, and the solvent is removed. The crude product is dissolved in methanol (20 ml), and camphorsulfonic acid (CSA) (100 mg) is added. The mixture is allowed to react over night. Evaporation of the solvent followed by silica gel chromatography (ethyl acetate/hexane 3/7) afforded 1085 mg (85%) of azido-alcohol. This intermediate (985 mg; 6.27 mmol) is dissolved in a dichloromethane/dimethylsulfoxide (DMSO) mixture (9:1,22 ml) and cooled to 0° C. Triethylamine (2.3 ml; 16.3 mmol; 2.6 equivalents) is added, followed by pyridine-SO₃ complexe (1000 mg; 12 mmol; 1.9 equivalents), and stirring continued for two hours at 0° C. The reaction mixture is poured onto brine, extracted twice with ethyl acetate, the organic layer is washed with brine, dried over sodium sulfate, and the solvent is removed. The residue is chromatographed on silica gel (ethyl acetate/hexane 25/75), and 690 mg (70%) of (R)-6-azido-5-methyl-hexanal is obtained as colorless oil.

IR (cm⁻¹): 2960; 2933; 2876; 2824; 2740; 2098; 1726; 1462; 1382; 1285. GC-MS: (M+H): 156

b) Preparation of (3S,5R)-1-[(Z,2E)—(R)-8-azido-7-methyl-oct-2-enylidene]-3,5-bis-(tert-butyl-dimethyl-silyloxy)-2-methylene-cydohexane

The Wittig reaction is carried out in analogy to reaction 1.1. f) starting with 730 mg (4.7 mmol) of aldehyde and 4.115 g (7.06 mmol) of phosphine oxide to yield 1780 mg (3S,5R)-1-[(Z,2E)—(R)-8-azido-7-methyl-oct-2-enylidene]-3,5-bis-(tert-butyl-dimethyl-silyloxy)-2-methylene-cydohexane.

IR (cm⁻¹): 2955; 2927; 2098; 1468; 1255; 1086; 912; 835; 780. MS: (M): 519

c) Preparation of a mixture of (R) and (S)—N-[(6E,8Z)—(R)-[(3S,5R)-3,5-dihydroxy-2-methylene-cyclohexylidene]-2-methyl-oct-6-enyl]-4,4,4-trifluoro-3-hydroxy-3-methyl-butyramide

(3S,5R)-1-[(Z,2E)—(R)-8-azido-7-methyl-oct-2-enylidene]-3,5-bis-(tert-butyl-dimethyl-silyloxy)-2-methylene-cydohexane (300 mg; 0.576 mmol) is dissolved in tetrahydrofuran/water (95:5; 5 ml), triphenylphosphine (165 mg; 0.63 mmol; 1.1 equivalents) is added and the reaction allowed to proceed over night. The solvents are then evaporated and the residual oil is dissolved in dichloromethane (10 ml). Dimethylaminopyridine (45 mg; 0.36 mmol; 0.6 equivalent), dicydohexyl-carbodiimide (220 mg; 1.07 mmol; 1.85 mmol), triethylamine (0.4 ml; 2.87 mmol; 5 equivalents), and 4,4,4-trifluoro-3-hydroxy-3-methyl-butyric acid (140 mg; 0.81 mmol; 1.4 equivalent) are added and the mixture is stirred for 20 hours. The reaction mixture is poured onto brine, extracted twice with ethyl acetate, the organic phase is washed with brine, dried over sodium sulfate and the solvent is removed. The residue is chromatographed on silica gel (ethyl acetate/hexane 2/8) to yield 122 mg of the protected amide.

This intermediate (120 mg, 0.185 mmol) is dissolved in tetrahydrofuran (5 ml), TBAF (4 ml of a 1M solution; 4 mmol; 20 equivalents) is added, and the mixture is stirred over night.

The reaction mixture is poured onto brine, extracted twice with ethyl acetate, the organic phase is washed with brine, dried over sodium sulfate and the solvent is removed. The residue is chromatographed on silica gel (isopropanol/hexane 25/75) and 40 mg (overall 16%) of a mixture of (R) and (S)—N-[(6E,8Z)—(R)-[(3S,5R)-3,5-dihydroxy-2-methylene-cyclohexylidene]-2-methyl-oct-6-enyl]-4,4,4-trifluoro-3-hydroxy-3-methyl-butyramide is obtained as colorless foam.

IR (cm⁻¹):3325; 2955; 1647; 1568; 1446; 1154; 1097; 1064; 987; 973; 926. MS: (M): 419

3.2. Preparation of N-[(6E,8Z)-(S)-[(3S,5R)-3,5-dihydroxy-2-methylene-cyclohexylidene]-2-methyl-oct-6-enyl]-4-isobutyramide

This compound is prepared in an analogous manner to the mixture of (R) and (S)—N-[(6E,8Z)—(R)-[(3S,5R)-3,5-dihydroxy-2-methylene-cyclohexylidene]-2-methyl-oct-6-enyl]-4,4,4-trifluoro-3-hydroxy-3-methyl-butyramide, described in Example 3.1, but using isobutyric acid as acyl component in step c, first paragraph.

IR (cm⁻¹): 3301; 2963; 2926; 2874; 1646; 1550; 1459; 1434; 1245; 1051; 976; 958; 910. MS: (M): 335

3.3. Preparation of a mixture of (R) and (S)—N-[(6E,8Z)-(S)-[(3S,5R)-3,5-dihydroxy-2-methylene-cyclohexylidene]-2-methyl-oct-6-enyl]-4,4,4-trifluoro-3-hydroxy-3-methyl-butyramide

This compound is obtained following the procedure used in Example 3.1. via (3S,5R)-1-[(Z,2E)-(S)-8-azido-7-methyl-oct-2-enylidene]-3,5-bis-(tert-butyl-dimethyl-silyloxy)-2-methylene-cyclohexane.

IR (cm⁻¹): 3330; 2952; 1649; 1570; 1446; 1154; 1097; 1064; 987; 973; 926. MS: (M): 419

The compounds of formula I can be administered orally, for the treatment of psoriasis, basal cell carcinomas, disorders of keratinization and keratosis; neoplastic diseases; disorders of the sebaceous glands such as acne and seborrhoic dermatitis, to hosts which need such treatment. More specifically, the compounds of formula I can be administered orally to a human in dosages that are in the range of 0.01 to 3 mg per day for the treatment of the above mentioned diseases.

The compounds of formula I can also be administered topically, for the treatment of psoriasis, basal cell carcinomas, disorders of keratinization and keratosis; neoplastic diseases; disorders of the sebaceous glands such as acne and seborrhoic dermatitis, to hosts which need such treatment. More specifically, the compounds of formula I can be administered topically to a human in dosages that are in the range of 0.01 to 3 mg per gram of topical formulation per day for the treatment of above-mentioned diseases.

The compounds of formula I can also be administered orally or topically for reversing the conditions associated with photodamage.

The dosage of the compounds of formula I can vary within wide limits depending on the illness to be treated, the age and the individual condition of the patient and on the mode of administration and will, of course, be fitted to the individual requirements in each particular case.

The invention is thus concerned with the use of the compounds of formula I for the treatment and prevention of hyperproliferative skin diseases such as psoriasis, basal cell carcinomas, disorders of keratinization and keratosis; neoplastic diseases; disorders of the sebaceous glands such as acne and seborrhoic dermatitis as well as for reversing to conditions associated with photodamage. The invention also relates to a method for treating and preventing said conditions by administering to a mammal a therapeutically active amount of a compound of formula I.

A “therapeutically effective amount” means the amount of a compound that, when administered to a mammal for treating or preventing a disease, is sufficient to effect such treatment or prevention for the disease. The “therapeutically effective amount” will vary depending on the compound, the disease and its severity and the age, weight, etc., of the mammal to be treated.

The pharmacological properties of the compounds of the formula I can be determined by the following test procedures:

Calcium Liability (Tolerance Test in Mice):

This test gives a global picture of calcemic liability. Profound changes in calcium homeostasis strongly affect the weight development of the animals. This parameter was used as a primary test for tolerance. Mice (25-30 g body weight) received daily subcutaneous administrations of the vitamin D derivative for 4 consecutive days. Body weight was registered just before and at the end of a 5-day treatment period. The “highest tolerated dose” (HTD_{sc}) in mice is the dose which results in zero weight gain during this treatment period.

The following compounds of formula I were tested:

A (1R,3R)-5-[(E)—(R)-7-(4-ethyl-4-hydroxy-hexyloxy)-oct-2-enylidene]-cyclohexane 1,3-diol

B (Z)-(1R,3S)-5-[(E)—(R)-9-[2-(1-hydroxy-1-methyl-ethyl)-phenyl]-7-methyl-non-2-enylidene]-4-methylene-cydohexane-1,3-diol

C (1R,3R)-5-[(E)—(R)-9-[2-(1-hydroxy-1-methyl-ethyl)-phenyl]-7-methyl-non-2-enylidene]-cydohexane-1,3-diol

D (Z)-(1R,3S)-5-[(E)—(R)-9-[2-(1-hydroxy-1-methyl-ethyl)-phenyl]-7-methyl-non-2-en-8-ynylidene]-4-methylene-cyclohexane-1,3-diol

The results are compiled in Table I below

| Compound | HTDs.c. |
|---|---|
| calcitriol | 1 |
| A | >5000 |
| B | >5000 |
| C | >4600 |
| D | >4800 |

HTDs.c. highest tolerated dose (μg/kg) without weight loss

No adverse effects for compounds of formula I were noted at the effective dose.

Oral dosage forms comprising compounds of formula I may be incorporated in capsules, tablets and the like with pharmaceutically acceptable carrier materials. Illustrative of such carrier materials which may be incorporated into capsules, and the like are the following: an emulsifier such as polyethylene glycol; a solubilizer such as a short chain triglyceride, e.g. Miglyol®; a binder such as gum tragacanth, acacia, corn starch, or gelatine; an excipient such as dicalcium phosphate; a disintegrating agent such as corn starch, potato starch or algenic acid; a lubricant such as magnesium stearate, a sweetening agent such as sucrose, lactose, or saccharin; a flavouring agent such as peppermint, oil of wintergreen or cherry. Various other materials may be present as coating or to otherwise modify the physical form of the dosage unit. For instance, tablets may be coated with shellac, sugar, or both. A syrup or elixir may contain the active compound, sucrose as a sweetening agent, methyl and propyl parabens as preservatives, a dye, and a flavoring such as cherry or orange flavor.

Topical dosage forms comprising compounds of formula I include: ointments and creams encompassing formulations having oleaginous, absorbable, water-soluble and emulsion-type bases such as petrolatum, lanolin, polyethylene glycols and the like. Lotions are liquid preparations and vary from simple solutions to aqueous or hydroalcoholic preparations containing finely divided substances. Lotions can contain suspending or dispersing agents, for example, cellulose derivatives such as ethyl cellulose, methyl cellulose, and the like; gelatin or gums, which incorporate the active ingredient in a vehicle made up of water, alcohol, glycerin and the like. Gels are semi-solid preparations made by gelling a solution or suspension of the active ingredient in a carrier vehicle. The vehicles, which can be hydrous or anhydrous, are gelled using a gelling agent, such as, carboxy polymethylene, and neutralized to a proper gel consistency with the use of alkalies, such as, sodium hydroxide and amines, such as, polyethylenecocoamine.

As used herein, the term “topical” denotes the use of the active ingredient, incorporated in a suitable pharmaceutical carrier, and applied at the site of the disorder for the exertion of local action. Accordingly, the topical composition includes those pharmaceutical forms in which compounds of formula I are applied externally by direct contact with the skin. The topical dosage forms comprise gels, creams, lotions, ointments, powders, aerosols and other conventional forms for applying medication to the skin obtained by admixing the compounds of formula I with known pharmaceutical topical carrier materials.

The following pharmaceutical compositions can be prepared in a manner known per se:

### Example A

Preparation of soft gelatine capsules

| Soft Gelatine Capsule | mg/Capsule |
|---|---|
| Active compound | 1 |
| Butylated hydroxytoluene (BHT) | 0.016 |
| Butylated hydroxyanisole (BHA) | 0.016 |
| Fractionated coconut oil (Neobee M-5) | 160.0 |
| or Miglyol 812 q.s. | |

### Example B

Preparation of soft gelatine capsules

| Soft Gelatine Capsule | mg/Capsule |
|---|---|
| Active compound | 1 |
| α-Tocopherol | 0.016 |
| Miglyol 812 q.s. | 160.0 |

### Example C

Preparation of a topical cream

| Topical Cream | mg/g |
|---|---|
| Active compound | 3 |
| Cetyl alcohol | 1.5 |
| Stearyl alcohol | 2.5 |
| Span 60 (sorbitan monostearate) | 2.0 |
| Arlacel 165 (glyceryl monostearate | 4.0 |
| and polyoxyethylene glycol stearate blend) | |
| Tween 60 (polysorbate 60) | 1.0 |
| Mineral oil | 4.0 |
| Propylene glycol | 5.0 |
| Propylparaben | 0.05 |
| BHA | 0.05 |
| Sorbitol solution | 2.0 |
| Edetate disodium | 0.01 |
| Methylparaben | 0.18 |
| Distilled water | q.s. |

1. Compounds of formula I: 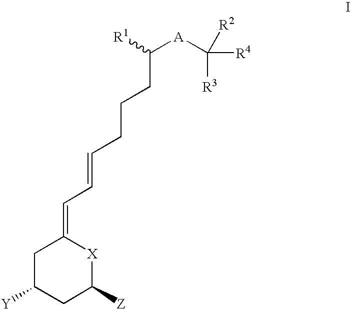
wherein
X is >C═CH₂ or —CH₂—;
Y and Z are independently of each other hydrogen, fluorine or hydroxy;
A is —O(CH₂)₃—, —(CH₂)₂-(1,2-C₆H₄)—, —CH═CH—(1,2-C₆H₄)—, —C≡C-(1,2-C₆H₄)—, —(CH₂)₂—CO—, —CH₂—O—CO—, —CH₂NHCO—, or —CH₂NHCOCH₂—;
R¹ is C₁-C₅-alkyl;
R² and R³ are independently of each other alkyl or perfluoroalkyl; and
R⁴ is hydrogen, hydroxy, C₁-C₅-alkyl or C₁-C₅-alkoxy.

2. The compounds according to claim 1 wherein at least one of Y and Z is hydroxy.

3. Compounds according to claims 1 or 2 wherein R⁴ is hydroxy.

4. Compounds according to claim 1 wherein Y and Z are both hydroxy.

5. Compounds according to claims 1 to 4, wherein A is a group —O(CH₂)₃—.

6. The compound according to claim 5 (1R,3R)-5-[(E)—(R)-7-(4-ethyl-4-hydroxy-hexyloxy)-oct-2-enylidene]-cydohexane 1,3-diol.

7. Compounds according to claims 1 to 4, wherein A is a group —CH₂NHCOCH₂— or —CH₂NHCO—.

8. The compounds according to claim 7
N-[(6E,8Z)-(S)-8-[(3S,5R)-3,5-Dihydroxy-2-methylene-cyclohexylidene]-2-methyl-oct-6-enyl]-isobutyramide;
mixture of (R)- and (S)—N-[(6E,8Z)—(R)-8-[(3S,5R)-3,5-dihydroxy-2-methylene-cyclohexylidene]-2-methyl-oct-6-enyl]-4,4,4-trifluoro-3-hydroxy-3-methyl-butyramide; and
mixture of (R)- and (S)—N-[(6E,8Z)-(S)-8-[(3S,5R)-3,5-dihydroxy-2-methylene-cyclohexylidene]-2-methyl-oct-6-enyl]-4,4,4-trifluoro-3-hydroxy-3-methyl-butyramide.

9. Compounds according to claims 1 to 4, wherein A is a group —(CH₂)₂-(1,2-C₆H₄)—.

10. The compounds according to claim 9
(Z)-(1R,3S)-5-[(E)—(R)-9-[2-(1-hydroxy-1-methyl-ethyl)-phenyl]-7-methyl-non-2-enylidene]-4-methylene-cyclohexane-1,3-diol; and
(1R,3R)-5-[(E)—(R)-9-[2-(1-hydroxy-1-methyl-ethyl)-phenyl]-7-methyl-non-2-enylidene]-cyclohexane-1,3-diol.

11. Compounds according to claims 1 to 4 wherein A is a group —C≡C-(1,2-C₆H₄)—.

12. The compound according to claim 11
(Z)-(1R,3S)-5-[(E)—(R)-9-[2-(1-hydroxy-1-methyl-ethyl)-phenyl]-7-methyl-non-2-en-8-ynylidene]-4-methylene-cyclohexane-1,3-diol.

13. The compounds according to claim 1 wherein A is a group —(CH₂)₂—CO—

14. The compounds according to claim 11
(E)—(R)-12-[(Z)-(3R,5R)-3,5-dihydroxy-2-methylene-cyclohexylidene]-2-hydroxy-2,6-dimethyl-dodec-10-en-3-one;
(E)—(R)-12-[(3R,5R)-3,5-dihydroxy-cyclohexylidene)-2-hydroxy-2,6-dimethyl-dodec-10-en-3-one; and
(E)—(R)-2-hydroxy-12-[(Z)-(S)-5-hydroxy-2-methylene-cyclohexylidene]-2,6-dimethyl-dodec-10-en-3-one.

15. Compounds according to claim 1 wherein A is a group —CH₂—O—CO— and R², R³ and R⁴ are alkyl.

16. The compound according to claim 15
2,2-dimethyl-propionic acid (2R)-8-((3R,5R)-3,5-dihydroxy-cyclohexylidene)-2-methyl-oct-6-enyl ester.

17. Pharmaceutical composition, particularly for the treatment or prevention of hyperproliferative skin diseases, specially psoriasis, basal cell carcinomas, disorders of keratinization and keratosis; or for reversing the conditions associated with photodamage, comprising compounds of formula I as defined in claim 1 as active ingredient and pharmaceutically acceptable carriers.

18. A process for preparing compounds of formula I as defined in claim 1 which comprises cleaving the protecting group(s) Y′, Z′ and/or R^{4′} contained in the compounds of formula 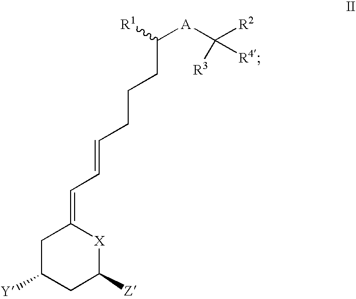
wherein Y′, Z′ are independently of each other hydrogen, fluorine or protected hydroxy groups and R^{4′} is hydrogen, C₁-C₅-alkyl, C₁-C₅-alkoxy or a protected hydroxy group, and R¹, R², R³, X and A are as defined in claim 1.

19. The compounds of anyone of claims 1 to 16 as therapeutic agents, especially as therapeutic agents for the treatment or prevention of hyperproliferative skin diseases, particularly psoriasis, basal cell carcinomas, disorders of keratinization and keratosis; or for reversing the conditions associated with photodamage.

20. The use of compounds of anyone of claims 1 to 16 for the manufacture of pharmaceutical compositions for the treatment or prevention of hyperproliferative skin diseases, particularly psoriasis, basal cell carcinomas, disorders of keratinization and keratosis; or for reversing the conditions associated with photodamage.

21. A method for treating or preventing hyperproliferative skin diseases, specially psoriasis, basal cell carcinomas, disorders of keratinization and keratosis in a mammal by administering to said mammal a therapeutically active amount of a compound according to any one of claims 1 to 16.

22. A method for reversing the conditions associated with photodamage in a mammal by administering to said mammal a therapeutically active amount of a compound according to any one of claims 1 to 16.

23. The compounds, formulations and methods as described herein.
